(19) United States
(12) Patent Application Publication
(10) Pub. No.: US 2023/0312777 A1
(43) Pub. Date: Oct. 5, 2023

(54) SEAL MEMBER FOR LIVESTOCK SENSOR AND LIVESTOCK SENSOR

(71) Applicant: DAIKIN INDUSTRIES, LTD., Osaka (JP)

(72) Inventors: Kenji ISHII, Osaka (JP); Yoshito TANAKA, Osaka (JP); Takumi SHIMOSUKI, Osaka (JP); Masaji KOMORI, Osaka (JP); Hirofumi MUKAE, Osaka (JP); Toshiyuki FUKUSHIMA, Osaka (JP); Akiyoshi YAMAUCHI, Osaka (JP); Yosuke KISHIKAWA, Osaka (JP); Hirokazu AOYAMA, Osaka (JP)

(73) Assignee: DAIKIN INDUSTRIES, LTD., Osaka (JP)

(21) Appl. No.: 18/330,632

(22) Filed: Jun. 7, 2023

Related U.S. Application Data
(63) Continuation of application No. PCT/JP2021/044384, filed on Dec. 3, 2021.

(30) Foreign Application Priority Data
Dec. 10, 2020 (JP) 2020-205068

(51) Int. Cl. C08F 14/26

(52) U.S. Cl. CPC C08F 14/26

(57) ABSTRACT

A seal for a livestock sensor containing a resin, or a rubber free from a chlorine atom. The seal is at least one selected from a packing, an O-ring, and a gasket, and the seal is configured to prevent a body fluid containing an organic acid in livestock from entering a space that contains a detecting portion or a substrate of the livestock sensor. Also disclosed is a livestock sensor including the seal.

### CROSS REFERENCE TO RELATED APPLICATIONS

This application is a Rule 53(b) Continuation of International Application No. PCT/JP2021/044384 filed Dec. 3, 2021, claiming priority based on Japanese Patent Application No. 2020-205068 filed Dec. 10, 2020, the respective disclosures of which are incorporated herein by reference in their entirety.

### TECHNICAL FIELD

The disclosure relates to seals for livestock sensors and livestock sensors.

### BACKGROUND ART

In the recent study in the field of livestock production, it has been considered to place sensors in the cattle's body to manage the health and breeding of cattle.

Patent Literature 1 discloses a detection device for detecting an internal state of the first stomach (rumen) of cattle, the detection device including an O-ring for preventing entry of rumen fluid.

### CITATION LIST

### Patent Literature

Patent Literature 1: WO 2010/147175

### SUMMARY

The disclosure relates to a seal for a livestock sensor, the seal containing a resin or a rubber free from a chlorine atom.

### Advantageous Effects

The disclosure can provide a seal for a livestock sensor that has excellent resistance to organic acids and excellently low acid permeability, and a livestock sensor including the same.

### BRIEF DESCRIPTION OF THE DRAWINGS

FIG. 1 is a cross-sectional view of an exemplary structure of a livestock sensor.

FIG. 2 is a cross-sectional diagram of a test device for inspecting chemical permeability.

### DESCRIPTION OF EMBODIMENTS

Livestock sensors to be orally administered to livestock (placed in the livestock body, particularly in the stomach) are required to be resistant to organic acids such as gastric acid.

Conventionally, sufficient study has not been made on the material of a seal used in such a livestock sensor.

As a result of intensive studies, the present inventors found that a seal containing a specific material has excellent resistance to organic acids and excellently low acid permeability and thus can be suitably used as a seal for a livestock sensor. The seal for a livestock sensor of the disclosure has been thus completed.

The disclosure will be specifically described below.

The seal for a livestock sensor of the disclosure contains a resin or a rubber free from a chlorine atom and thus has excellent resistance to organic acids and excellently low acid permeability. When used in a livestock sensor, the seal is less likely to corrode even when it contacts with gastric juice, for example and is not likely to allow permeation of gastric juice, for example, thereby preventing the gastric juice, for example, from entering the inside of the livestock sensor to avoid corrosion of a component such as an internal substrate.

Examples of the resin include a fluororesin and a silicone resin. A fluororesin is preferred as it has much better resistance to organic acids, much lower acid permeability, and a high specific gravity.

The fluororesin preferably has a melting point of 100° C. to 360° C., more preferably 140° C. to 350° C., still more preferably 160° C. to 340° C.

The melting point is the temperature corresponding to the maximum value on a heat-of-fusion curve obtained by increasing the temperature at a rate of 10° C./min using a differential scanning calorimeter (DSC).

The fluororesin preferably has a specific gravity of 1.8 or higher. The fluororesin having a specific gravity within the above range can increase the specific gravity of the livestock sensor, facilitating placement (submerging) of the sensor in a body fluid such as gastric juice. The specific gravity is more preferably 1.9 or higher, still more preferably 2.0 or higher. The upper limit of the specific gravity is preferably, but is not limited to, 3.0 or lower, more preferably 2.5 or lower.

The specific gravity of the fluororesin is measured in conformity with ASTM D-792.

Examples of the fluororesin include polytetrafluoroethylene (PTFE), a tetrafluoroethylene (TFE)/perfluoro(alkyl vinyl ether) (PAVE) copolymer (PFA), a TFE/hexafluoropropylene (HFP) copolymer (FEP), an ethylene (Et)/TFE copolymer (ETFE), an Et/TFE/HFP copolymer (EFEP), polychlorotrifluoroethylene (PCTFE), a chlorotrifluoroethylene (CTFE)/TFE copolymer, a CTFE/TFE/PAVE copolymer, an Et/CTFE copolymer, polyvinyl fluoride (PVF), polyvinylidene fluoride (PVdF), a vinylidene fluoride (VdF)/TFE copolymer, a VdF/HFP copolymer, a VdF/TFE/HFP copolymer, a VdF/HFP/(meth)acrylic acid copolymer, a VdF/CTFE copolymer, a VdF/pentafluoropropylene copolymer, a VdF/PAVE/TFE copolymer, and a TFE/perfluoroalkyl allyl ether copolymer. The perfluoroalkyl allyl ether is a monomer represented by CF₂═CFCF₂—O—Rf⁴ (wherein Rf⁴ is a C1-C5 perfluoroalkyl group).

The fluororesin is preferably a perhalopolymer as it has much better resistance to organic acids and much lower acid permeability. A perhalopolymer is a polymer in which every carbon atom constituting the main chain of the polymer is coupled with a halogen atom.

The fluororesin preferably includes at least one selected from the group consisting of PTFE, PFA, FEP, a TFE/perfluoroalkyl allyl ether copolymer, and a CTFE/TFE/PAVE copolymer, more preferably at least one selected from the group consisting of PTFE, PFA, and a CTFE/TFE/PAVE copolymer.

The PTFE may be a TFE homopolymer consisting only of a tetrafluoroethylene (TFE) unit, or may be a modified PTFE containing a TFE unit and a modifying monomer unit based on a modifying monomer copolymerizable with TFE.

The modifying monomer may be any monomer copolymerizable with TFE, and examples thereof include a perfluoroolefin such as hexafluoropropylene (HFP); a chlorofluoroolefin such as chlorotrifluoroethylene (CTFE); a hydrogen-containing fluoroolefin such as trifluoroethylene and vinylidene fluoride (VdF); a perfluorovinyl ether; a perfluoroalkyl allyl ether; a (perfluoroalkyl)ethylene; and ethylene. One type or two or more types of modifying monomers may be used.

The perfluorovinyl ether is not limited, and may be, for example, an unsaturated perfluoro compound represented by the following formula (1):

CF₂=CF—ORf (1)

wherein Rf is a perfluoroorganic group. The term “perfluoro organic group” herein means an organic group in which all hydrogen atoms bonded to any carbon atom are replaced by fluorine atoms. The perfluoro organic group may contain an ether oxygen.

Examples of the perfluorovinyl ether include a perfluoro(alkyl vinyl ether) (PAVE) represented by the formula (1) wherein Rf is a C1-C10 perfluoroalkyl group. The perfluoroalkyl group preferably has 1 to 5 carbon atoms.

Examples of the perfluoroalkyl group in the PAVE include a perfluoromethyl group, a perfluoroethyl group, a perfluoropropyl group, a perfluorobutyl group, a perfluoropentyl group, and a perfluorohexyl group. Preferred is perfluoro(propyl vinyl ether) (PPVE) wherein the perfluoroalkyl group is a perfluoropropyl group.

Examples of the perfluorovinyl ether further include: those represented by the formula (1) wherein Rf is a C4-C9 perfluoro(alkoxyalkyl) group, those represented by the formula (1) wherein Rf is represented by the following formula:

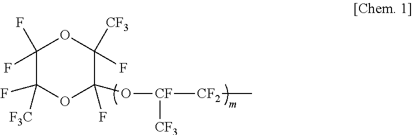

wherein m is 0 or an integer of 1 to 4; and those represented by the formula (1) wherein Rf is a group represented by the following formula:

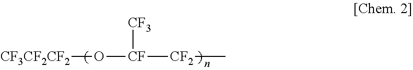

wherein n is an integer of 1 to 4.

Examples of the (perfluoroalkyl)ethylene include, but are not limited to, (perfluorobutyl)ethylene (PFBE), (perfluorohexyl)ethylene (PFHE), and (perfluorooctyl)ethylene.

The modifying monomer in the modified PTFE preferably includes at least one selected from the group consisting of HFP, CTFE, VdF, PPVE, PFBE, and ethylene, more preferably at least one selected from the group consisting of HFP and CTFE.

In the modified PTFE, the amount of the modifying monomer unit is preferably in the range of 0.00001 to 1.0% by mass. The lower limit of the amount of the modifying monomer unit is more preferably 0.0001% by mass, still more preferably 0.001% by mass, even more preferably 0.005% by mass, further preferably 0.010% by mass, particularly preferably 0.030% by mass. The upper limit of the amount of the modifying monomer unit is preferably 0.90% by mass, more preferably 0.50% by mass, still more preferably 0.40% by mass, even more preferably 0.30% by mass.

The term “modifying monomer unit” herein means a moiety that is part of the molecular structure of the modified PTFE and is derived from a modifying monomer. The term “all monomer units” herein means all moieties derived from monomers in the molecular structure of the modified PTFE.

The PTFE preferably has a melting point of 324° C. to 360° C. The melting point of PTFE means the first melting point. The first melting point is the temperature corresponding to the maximum value on a heat-of-fusion curve obtained by heating a PTFE that has no history of being heated up to a temperature of 300° C. or higher, at a rate of 10° C./min using a differential scanning calorimeter (DSC).

The PTFE preferably has a standard specific gravity (SSG) of 2.130 to 2.280. The standard specific gravity is more preferably 2.220 or lower, still more preferably 2.200 or lower, while it is preferably 2.140 or higher, more preferably 2.150 or higher. The SSG is measured by a water displacement method in conformity with ASTM D-792 using a sample molded in conformity with ASTM D 4895-89.

The PTFE preferably has non-melt secondary processibility. The non-melt secondary processibility means a property of a polymer such that the melt flow rate cannot be measured at a temperature higher than the crystallization melting point in conformity with ASTM D-1238 and D-2116.

The PFA is preferably, but is not limited to, a copolymer having a molar ratio of the TFE unit to the PAVE unit (TFE unit/PAVE unit) of 70/30 or more and less than 99/1, more preferably 70/30 or more and 98.9/1.1 or less, still more preferably 80/20 or more and 98.9/1.1 or less. The PFA is also preferably a copolymer containing 0.1 to 10 mol % (the sum of the TFE unit and the PAVE unit is 90 to 99.9 mol %), more preferably 0.1 to 5 mol %, particularly preferably 0.2 to 4 mol % of a monomer unit derived from a monomer copolymerizable with TFE and PAVE.

Examples of the monomer copolymerizable with TFE and PAVE include HFP, a vinyl monomer represented by the formula (I): CZ¹Z²=CZ³(CF₂)ₙZ⁴ (wherein Z¹, Z², and Z³ are the same as or different from each other and each are a hydrogen atom or a fluorine atom; Z⁴ is a hydrogen atom, a fluorine atom, or a chlorine atom; and n is an integer of 2 to 10), an alkyl perfluorovinyl ether derivative represented by the formula (II): CF₂=CF—OCH₂—Rf¹ (wherein Rf¹ is a C1-C5 perfluoroalkyl group), and an allyl ether monomer represented by the formula (X): CZ⁵Z⁶=CZ⁷—CZ⁶Z⁹—O—Rf⁴ (wherein Z⁵, Z⁶, and Z⁷ are the same as or different from each other and each represent a hydrogen atom, a chlorine atom, or a fluorine atom; Z⁸ and Z⁹ each are a hydrogen atom or a fluorine atom; and Rf⁴ is a C1-C5 perfluoroalkyl group). Examples of the allyl ether monomer include CH₂═CFCF₂—O—Rf⁴, CF₂═CFCF₂—O—Rf⁴ (perfluoroalkyl allyl ether), CF₂═CFCH₂—O—Rf⁴, and CH₂═CHCF₂—O—Rf⁴ (in the formulas, Rf⁴ is the same as that in the formula (X)).

Examples of the monomer copolymerizable with TFE and PAVE further include unsaturated monocarboxylic acids, unsaturated dicarboxylic acids, and acid anhydrides of unsaturated dicarboxylic acids, such as itaconic acid, itaconic anhydride, citraconic anhydride, and 5-norbornene-2,3-dicarboxylic anhydride.

The PFA preferably has a melting point of 180° C. or higher and lower than 324° C., more preferably 230° C. to 320° C., still more preferably 280° C. to 320° C.

The FEP is preferably, but is not limited to, a copolymer having a molar ratio of the TFE unit to the HFP unit (TFE unit/HFP unit) of 70/30 or more and less than 99/1, more preferably 70/30 or more and 98.9/1.1 or less, still more preferably 80/20 or more and 98.9/1.1 or less. The FEP is also preferably a copolymer containing 0.1 to 10 mol % (the sum of the TFE unit and the HFP unit is 90 to 99.9 mol %), more preferably 0.1 to 5 mol %, particularly preferably 0.2 to 4 mol % of a monomer unit derived from a monomer copolymerizable with TFE and HFP.

Examples of the monomer copolymerizable with TFE and HFP include PAVE, a monomer represented by the formula (X), and an alkyl perfluorovinyl ether derivative represented by the formula (II).

Examples of the monomer copolymerizable with TFE and HFP further include unsaturated monocarboxylic acids, unsaturated dicarboxylic acids, acid anhydrides of unsaturated dicarboxylic acids, such as itaconic acid, itaconic anhydride, citraconic anhydride, and 5-norbornene-2,3-dicarboxylic anhydride.

The FEP preferably has a melting point of 150° C. or higher and lower than 324° C., more preferably 200° C. to 320° C., still more preferably 240° C. to 320° C.

The CTFE/TFE/PAVE copolymer (CPT) is a copolymer consisting essentially of CTFE, TFE, and PAVE.

The PAVE in the CTFE/TFE/PAVE copolymer may be perfluoro(methyl vinyl ether) (PMVE), perfluoro(ethyl vinyl ether) (PEVE), perfluoro(propyl vinyl ether) (PPVE), perfluoro(butyl vinyl ether), or the like, and preferably includes at least one selected from the group consisting of PMVE, PEVE, and PPVE.

The CTFE/TFE/PAVE copolymer preferably contains the PAVE unit in an amount of 0.5 mol % or more and 5 mol % or less of all monomer units.

The values of constituent units such as a CTFE unit are obtainable by ¹⁹F-NMR analysis.

The CTFE/TFE/PAVE copolymer preferably has a melting point of 160° C. to 270° C.

The amounts of the respective monomer units in the polymer described above can be calculated by appropriate combination of NMR, FT-IR, elemental analysis, and X-ray fluorescence analysis in accordance with the types of the monomers.

The fluororesin is also preferably a melt-fabricable fluororesin. Use of a melt-fabricable fluororesin improves processibility.

The term “melt-fabricable” herein means that the polymer can be melted and processed using a conventional processor such as an extruder or an injection molding machine.

The melt-fabricable fluororesin preferably has a melt flow rate (MFR) of 0.1 to 100 g/10 min, more preferably 0.5 to 50 g/10 min.

The MFR herein is a value obtained in conformity with ASTM D1238 using a melt indexer, as the mass (g/10 min) of a polymer flowing out of a nozzle (inner diameter: 2 mm, length: 8 mm) per 10 minutes at a measurement temperature specified according to the type of the fluoropolymer (e.g., 372° C. for PFA and FEP, 297° C. for CPT and ETFE) and a load specified according to the type of the fluoropolymer (e.g., 5 kg for PFA, FEP, CPT, and ETFE).

Examples of the melt-fabricable fluororesin include those mentioned above, including PFA, FEP, ETFE, EFEP, PCTFE, a CTFE/TFE/PAVE copolymer, and PVdF. The melt-fabricable fluororesin preferably includes at least one selected from the group consisting of PFA, FEP, and a CTFE/TFE/PAVE copolymer.

The rubber free from a chlorine atom is preferably amorphous. The term “amorphous” means that the rubber has a melting peak (ΔH) of 2.0 J/g or lower determined by DSC (temperature-increasing rate: 10° C./min).

Examples of the rubber free from a chlorine atom include fluorine-containing rubbers (excluding those containing chlorine atoms) such as a fluororubber and a fluorosilicone rubber, fluorine-free rubbers free from a chlorine atom.

Examples of the fluororubber include a partially fluorinated rubber and a perfluororubber. A perfluororubber as it has much better resistance to organic acids and much lower acid permeability is preferred.

Examples of the partially fluorinated rubber include a vinylidene fluoride (VdF)-based fluororubber, a tetrafluoroethylene (TFE)/propylene (Pr)-based fluororubber, a tetrafluoroethylene (TFE)/propylene/vinylidene fluoride (VdF)-based fluororubber, an ethylene/hexafluoropropylene (HFP)-based fluororubber, an ethylene/hexafluoropropylene (HFP)/vinylidene fluoride (VdF)-based fluororubber, and an ethylene/hexafluoropropylene (HFP)/tetrafluoroethylene (TFE)-based fluororubber. Preferred among these is at least one selected from the group consisting of a vinylidene fluoride-based fluororubber and a tetrafluoroethylene/propylene-based fluororubber.

The vinylidene fluoride-based fluororubber is preferably a copolymer containing 45 to 85 mol % of vinylidene fluoride and 55 to 15 mol % of at least one different monomer copolymerizable with vinylidene fluoride. Preferred is a copolymer containing 50 to 80 mol % of vinylidene fluoride and 50 to 20 mol % of at least one different monomer copolymerizable with vinylidene fluoride.

The amounts of the respective monomers constituting the fluoropolymer herein can be calculated by appropriate combination of NMR, FT-IR, elemental analysis, and X-ray fluorescence analysis in accordance with the types of the monomers.

Examples of the at least one different monomer copolymerizable with vinylidene fluoride includes tetrafluoroethylene (TFE), hexafluoropropylene (HFP), a fluoroalkyl vinyl ether, trifluoroethylene, tri fluoropropylene, pentafluoropropylene, trifluorobutene, tetrafluoroisobutene, hexafluoroisobutene, vinyl fluoride, a perfluoroalkyl allyl ether, a fluoromonomer represented by the formula (6): CH₂=CFRf⁶¹ (wherein Rf⁶¹ is a C1-C12 linear or branched fluoroalkyl group), a fluoromonomer represented by the formula (7): CH₂═CH—(CF₂)ₙ—X² (wherein X² is H or F and n is an integer of 3 to 10), a monomer giving a crosslinking site; and non-fluorinated monomers such as ethylene, propylene, and an alkyl vinyl ether. Each of these can be used alone or in any combination. Among these, at least one selected from the group consisting of TFE, HFP, and a fluoroalkyl vinyl ether is preferably used.

The fluoroalkyl vinyl ether preferably includes at least one selected from the group consisting of a fluoromonomer represented by the formula (8):

CF₂=CF—ORf⁸¹

(wherein Rf⁸¹ is a C1-C8 perfluoroalkyl group), a fluoromonomer represented by the formula (9):

CF₂=CFOCF₂ORf⁹¹

(wherein Rf⁹¹ is a C1-C6 linear or branched perfluoroalkyl group, a C5-C6 cyclic perfluoroalkyl group, or a C2-C6 linear or branched perfluorooxyalkyl group containing 1 to 3 oxygen atom(s)), and a fluoromonomer represented by the formula (10):

CF₂=CFO(CF₂CF(Y¹⁰)O)ₘ(CF₂)ₙF

(wherein Y¹⁰ is a fluorine atom or a trifluoromethyl group, m is an integer of 1 to 4, and n is an integer of 1 to 4). More preferred is a fluoromonomer represented by the formula (8).

The perfluoroalkyl allyl ether is preferably a fluoromonomer represented by the formula (11): CF₂=CF—CF₂ORf¹¹¹ (wherein Rf¹¹¹ is a C1-C5 perfluoroalkyl group).

Specific examples of the vinylidene fluoride-based fluororubber include a VdF/HFP-based rubber, a VdF/HFP/TFE-based rubber, a rubber based on VdF and a fluoromonomer rubber represented by the formula (6), a rubber based on VdF, a fluoromonomer represented by the formula (6), and TFE, a VdF/perfluoro(methyl vinyl ether) (PMVE)-based rubber, a VdF/PMVE/TFE-based rubber, and a VdF/PMVE/TFE/HFP-based rubber. The rubber based on VdF and a fluoromonomer represented by the formula (6) is preferably VdF/CH₂═CFCF₃ rubber. The rubber based on VdF, a fluoromonomer represented by the formula (6), and TFE is preferably VdF/TFE/CH₂═CFCF₃ rubber.

The VdF/CH₂═CFCF₃-based rubber is preferably a copolymer containing 40 to 99.5 mol % of VdF and 0.5 to 60 mol % of CH₂═CFCF₃, more preferably a copolymer containing 50 to 85 mol % of VdF and 20 to 50 mol % of CH₂═CFCF₃.

The tetrafluoroethylene/propylene-based fluororubber is preferably a copolymer containing 45 to 70 mol % of tetrafluoroethylene, 55 to 30 mol % of propylene, and 0 to 5 mol % of a fluoromonomer giving a crosslinking site.

The perfluororubber preferably includes at least one selected from the group consisting of perfluororubbers containing TFE, such as a copolymer of TFE/a fluoromonomer represented by the formula (8), (9), or (10) and a copolymer of TFE/a fluoromonomer represented by the formula (8), (9), or (10)/a monomer giving a crosslinking site.

In the case of a TFE/PMVE copolymer, the compositional ratio is preferably (45 to 90)/(10 to 55) (mol %), more preferably (55 to 80)/(20 to 45), still more preferably (55 to 70)/(30 to 45).

In the case of a copolymer of TFE/PMVE/a monomer giving a crosslinking site, the compositional ratio is preferably (45 to 89.9)/(10 to 54.9)/(0.01 to 4) (mol %), more preferably (55 to 77. 9)/(20 to 49.9)/(0.1 to 3.5), still more preferably (55 to 69.8)/(30 to 44.8)/(0.2 to 3).

In the case of a copolymer of TFE/a C4-C12 fluoromonomer represented by the formula (8), (9), or (10), the compositional ratio is preferably (50 to 90)/(10 to 50) (mol %), more preferably (60 to 88)/(12 to 40), still more preferably (65 to 85)/(15 to 35).

In the case of a copolymer of TFE/a C4-C12 fluoromonomer represented by the formula (8), (9), or (10)/a monomer giving a crosslinking site, the compositional ratio is preferably (50 to 89.9)/(10 to 49.9)/(0.01 to 4) (mol %), more preferably (60 to 87.9)/(12 to 39.9)/(0.1 to 3.5), still more preferably (65 to 84.8)/(15 to 34.8)/(0.2 to 3).

If the compositional ratios are out of the ranges mentioned above, the copolymers tend to lose the properties as a rubber elastic body and to have the properties similar to those of resin.

The perfluororubber preferably includes at least one selected from the group consisting of a copolymer of TFE/a fluoromonomer represented by the formula (10)/a fluoromonomer giving a crosslinking site, a copolymer of TFE/a perfluorovinyl ether represented by the formula (10), a copolymer of TFE/a fluoromonomer represented by the formula (8), and a copolymer of TFE/a fluoromonomer represented by the formula (8)/a monomer giving a crosslinking site.

Examples of the perfluororubber include the perfluororubbers disclosed in WO 97/24381, JP S61-57324 B, JP H4-81608 B, and JP H5-13961 B.

The monomer giving a crosslinking site is a monomer (cure-site monomer) containing a crosslinkable group that can give a fluoropolymer a crosslinking site to form a crosslink with use of a crosslinking agent.

The monomer giving a crosslinking site preferably includes at least one selected from the group consisting of:

a fluoromonomer represented by the formula (12):

CX³₂═CX³—R_{f}¹²¹CHR¹²¹X₄

(wherein X³ is a hydrogen atom, a fluorine atom, or CH₃; R_{f}¹²¹ is a fluoroalkylene group, a perfluoroalkylene group, a fluoro(poly)oxyalkylene group, or a perfluoro(poly)oxyalkylene group; R¹²¹ is a hydrogen atom or CH₃; and X⁴ is an iodine atom or a bromine atom);

a fluoromonomer represented by the formula (13):

CX³₂═CX³—R_{f}¹³¹X⁴

(wherein X³ is a hydrogen atom, a fluorine atom, or CH₃; R_{f}¹³¹ is a fluoroalkylene group, a perfluoroalkylene group, a fluoropolyoxyalkylene group, or a perfluoropolyoxyalkylene group; and X⁴ is an iodine atom or a bromine atom);

a fluoromonomer represented by the formula (14):

CF₂=CFO(CF₂CF(CF₃)O)ₘ(CF₂)ₙ-X⁵

(wherein m is an integer of 0 to 5; n is an integer of 1 to 3; and X⁵ is a cyano group, a carboxy group, an alkoxycarbonyl group, an iodine atom, a bromine atom, or —CH₂I);

a fluoromonomer represented by the formula (15):

CH₂═CFCF₂O(CF(CF₃)CF₂O)ₘ(CF(CF₃))ₙ-X⁶

(wherein m is an integer of 0 to 5; n is an integer of 1 to 3; X⁶ is a cyano group, a carboxy group, an alkoxycarbonyl group, an iodine atom, a bromine atom, or —CH₂OH), and

a monomer represented by the formula (16):

CR¹⁶²R¹⁶³═CR¹⁶⁴—Z—CR¹⁶⁵═CR¹⁶⁶R¹⁶⁷

(wherein R¹⁶², R¹⁶³, R¹⁶⁴, R¹⁶⁵, R¹⁶⁶, and R¹⁶⁷ are the same as or different from each other, and are each a hydrogen atom or a C1-C5 alkyl group; Z is a C1-C18 linear or branched alkylene group optionally containing an oxygen atom, a C3-C18 cycloalkylene group, a C1-C10 alkylene or oxyalkylene group that is at least partially fluorinated, or a (per) fluoropolyoxyalkylene group represented by -(Q)ₚ-CF₂O—(CF₂CF₂O)ₘ(CF₂O)ₙ—CF₂-(Q)ₚ- (wherein Q is an alkylene group or an oxyalkylene group; p is 0 or 1; and m/n is 0.2 to 5) and having a molecular weight of 500 to 10000).

X³ is preferably a fluorine atom. R_{f}¹²¹ and R_{f}¹³¹ are preferably C1-C5 perfluoroalkylene groups. R¹²¹ is preferably a hydrogen atom. X⁵ is preferably a cyano group, an alkoxycarbonyl group, an iodine atom, a bromine atom, or —CH₂I. X⁶ is preferably a cyano group, an alkoxycarbonyl group, an iodine atom, a bromine atom, or —CH₂OH.

The monomer giving a crosslinking site preferably includes at least one selected from the group consisting of CF₂=CFOCF₂CF(CF₃) OCF₂CF₂CN, CF₂=CFOCF₂CF(CF₃) OCF₂CF₂COOH, CF₂=CFOCF₂CF(CF₃) OCF₂CF₂CH₂I, CF₂=CFOCF₂CF₂CH₂I, CH₂═CFCF₂OCF(CF₃) CF₂OCF CF₃CN, CH₂═CFCF₂OCF(CF₃) CF₂OCF(CF₃) COOH, CH₂═CFCF₂OCF(CF₃) CF₂OCF(CF₃) CH₂OH, CH₂=CHCF₂CF₂I, CH₂═CH(CF₂)₂CH═CH₂, CH₂═CH(CF₂) ₆CH═CH₂, and CF₂═CFO(CF₂) ₅CN, more preferably at least one selected from the group consisting of CF₂=CFOCF₂CF(CF₃)OCF₂CF₂CN and CF₂=CFOCF₂CF₂CH₂I.

In order to achieve excellent compression set performance at high temperature, the fluororubber preferably has a glass transition temperature of −70° C. or higher, more preferably −60° C. or higher, still more preferably −50° C. or higher. In order to achieve good cold resistance, the fluororubber has a glass transition temperature of preferably 5° C. or lower, more preferably 0° C. or lower, still more preferably −3° C. or lower.

The glass transition temperature can be determined as follows. Specifically, using a differential scanning calorimeter (DSC822e available from Mettler-Toledo International Inc.), 10 mg of a sample is heated at a rate of 10° C./min to give a DSC curve, and the temperature is read at the intermediate point of two intersections between each of the extension lines of the base lines before and after the secondary transition of the DSC curve and the tangent line at the inflection point of the DSC curve.

In order to achieve good heat resistance, the fluororubber preferably has a Mooney viscosity ML(1+20) at 170° C. of 30 or higher, more preferably 40 or higher, still more preferably 50 or higher. In order to achieve good processibility, the fluororubber preferably has a Mooney viscosity ML(1+20) at 170° C. of 150 or lower, more preferably 120 or lower, still more preferably 110 or lower.

In order to achieve good heat resistance, the fluororubber preferably has a Mooney viscosity ML(1+20) at 140° C. of 30 or higher, more preferably 40 or higher, still more preferably 50 or higher. In order to achieve good processibility, the fluororubber preferably has a Mooney viscosity ML(1+20) at 140° C. of 180 or lower, more preferably 150 or lower, still more preferably 110 or lower.

In order to achieve good heat resistance, the fluororubber preferably has a Mooney viscosity ML(1+10) at 100° C. of 10 or higher, more preferably 20 or higher, still more preferably 30 or higher. In order to achieve good processibility, the fluororubber preferably has a Mooney viscosity ML(1+10) at 100° C. of 120 or lower, more preferably 100 or lower, still more preferably 80 or lower.

The Mooney viscosity can be determined using a Mooney viscometer MV2000E available from Alpha Technologies Inc. at 170° C., 140° C., or 100° C. in conformity with JIS K 6300.

Examples of the fluorine-free rubbers free from a chlorine atom include nitrile rubber, hydrogenated nitrile rubber, styrene-butadiene rubber, polybutadiene rubber, natural rubber, isoprene rubber, ethylene-α-olefin rubber, ethylene-α-olefin-nonconjugated diene rubber, acrylic rubber, ethylene acrylic rubber, silicone rubber, butyl rubber, ethylene-vinyl ester rubber, and ethylene-methacrylate rubber. Preferred among these are nitrile rubber and silicone rubber.

In order to achieve much better resistance to organic acids and much lower acid permeability as well as a high specific gravity, the rubber free from a chlorine atom is preferably a fluorine-containing rubber, more preferably a fluororubber, still more preferably a perfluororubber.

The seal of the disclosure may further contain a different component in addition to the resin or rubber. Examples of the different component include various additives such as fillers (e.g., carbon black, barium sulfate), acid acceptors, processing aids (e.g., wax), plasticizers, colorants, stabilizers, adhesion aids, release agents, conductivity-imparting agents, thermal-conductivity-imparting agents, surface non-adhesive agents, flexibility-imparting agents, heat resistance improvers, and flame retardants. The amount of the different component is preferably 0 to 50 parts by mass, more preferably 0 to 20 parts by mass, still more preferably 0 to 10 parts by mass per 100 parts by mass of the resin or rubber.

The seal of the disclosure can be produced by molding the resin by a known molding method such as cutting, injection molding, extrusion molding, or compression molding. The seal of the disclosure can also be produced by crosslink-molding a rubber composition containing the rubber and a crosslinking agent (and a crosslinking aid, if necessary) by a known method.

The seal of the disclosure is used in a livestock sensor to prevent entry of a fluid (liquid or gas) or a foreign substance, preferably a fluid, from the outside. The fluid is preferably a liquid, more preferably a body fluid containing an organic acid in livestock, still more preferably livestock gastric juice, particularly preferably livestock rumen fluid.

The seal preferably prevents a fluid or a foreign substance from entering a space that contains a detecting portion or a substrate of the livestock sensor.

The form of the seal is not limited, and can be determined according to the application site. Examples thereof include a packing, an O-ring, and a gasket.

A livestock sensor to which the seal of the disclosure is applied is placed in the livestock body and detects the state of the livestock (e.g., pH, temperature, amount of exercise (acceleration)). The livestock sensor is preferably configured to be orally administered to livestock. Further, the livestock sensor is preferably a wireless transmission sensor capable of wirelessly transmitting acquired data.

The disclosure also relates to a livestock sensor including the above-described seal of the disclosure. When including the seal of the disclosure, the livestock sensor of the disclosure is less likely to deteriorate even when it contacts with gastric juice, for example, and is not likely to allow permeation of gastric juice, for example, thereby preventing the gastric juice, for example, from entering the inside to avoid corrosion of a component such as an internal substrate.

The livestock sensor preferably includes a housing. The housing is preferably a member capable of containing a detecting portion and other necessary parts inside. Also, the housing of the disclosure may be configured such that a portion thereof can be separated (for example, the main body and the cap).

The housing may be formed of any material, including resins such as a fluororesin or a fluorine-free resin; or metals such as stainless steel. The outer surface of a metal housing may be coated with resin.

The housing may have any shape that can contain the detecting portion and other necessary parts inside, such as a tubular shape (e.g., circular tube, square tube), a bottle shape, a bottomed circular tubular shape, or a bottomed square tubular shape. Preferred among these are a tubular shape, a bottle shape, a bottomed circular tubular shape, and a bottomed square tubular shape, and more preferred are a circular tubular shape and a bottomed circular tubular shape.

The livestock sensor preferably includes a detecting portion contained in the housing. Examples of the detecting portion include a pH sensor, a temperature sensor, a piezoelectric sensor, an acceleration sensor, and a position sensor.

The livestock are preferably ruminant animals including cattle (dairy cattle, beef cattle), sheep, and goats. Cattle are particularly preferred.

The livestock sensor is preferably placed in an internal organ of livestock, more preferably in the stomach, still more preferably in the rumen, particularly preferably in the rumen fluid.

The livestock sensor is preferably left in the livestock body for one month or longer, more preferably six months or longer, still more preferably one year or longer, particularly preferably three years or longer.

The livestock sensor preferably has a specific gravity of 1.8 or higher, more preferably 2.0 or higher. The livestock sensor having a specific gravity within the above range can be easily placed (submerged) in a body fluid such as gastric juice.

The livestock sensor may have any size that allows oral administration to livestock. In the case of the circular tubular sensor, the diameter may be 10 to 35 mm and the length may be 40 to 150 mm, for example.

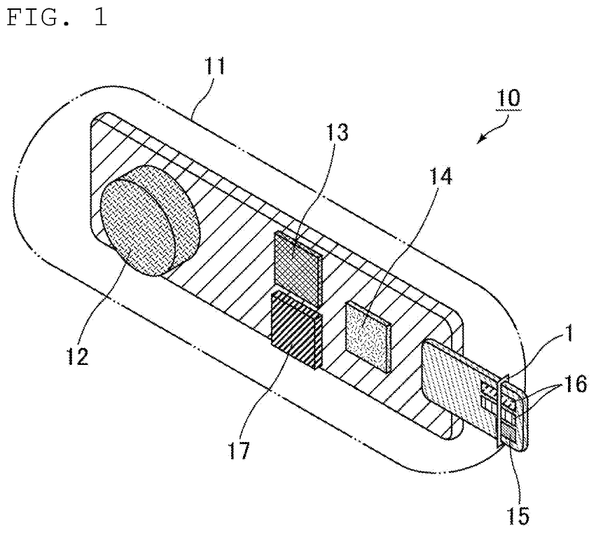

FIG. 1 shows an exemplary structure of the livestock sensor of the disclosure. The livestock sensor of the disclosure is not limited to this.

A livestock sensor 10 in FIG. 1 includes a housing 11. The housing 11 contains a signal processing circuit 13 connected to a battery 12. The signal processing circuit 13 is provided with an acceleration sensor 14 and a radio transmitter 17. To the signal processing circuit 13 are electrically connected a temperature sensor 15 and a fixed pH sensor 16.

The temperature sensor 15 and the fixed pH sensor 16 are partly exposed outside the housing 11 so as to contact with the rumen fluid.

The housing 11 is provided with a packing 1 to prevent rumen fluid from flowing into the space containing the signal processing circuit 13. The packing 1 corresponds to the seal of the disclosure.

The disclosure relates to a seal for a livestock sensor, the seal containing a resin or a rubber free from a chlorine atom.

The resin is preferably a fluororesin.

The fluororesin preferably includes at least one selected from the group consisting of polytetrafluoroethylene, a tetrafluoroethylene/perfluoro(alkyl vinyl ether) copolymer, a tetrafluoroethylene/hexafluoropropylene copolymer, a tetrafluoroethylene/perfluoroalkyl allyl ether copolymer, and a chlorotrifluoroethylene/tetrafluoroethylene/perfluoro(alkyl vinyl ether) copolymer.

The rubber is preferably a fluorine-containing rubber.

The fluorine-containing rubber is preferably a perfluororubber.

The disclosure also relates to a livestock sensor including the seal for a livestock sensor.

### EXAMPLES

The disclosure will be described in more detail with reference to examples, but the disclosure is not limited only to these examples.

The materials used in the experimental examples are shown below.
PTFE: TFE homopolymer (melting point: 327° C., SSG: 2.2)
PFA: TFE/PPVE copolymer (melting point: 306° C., MFR: 14 g/10 min)
CPT: CTFE/TFE/PPVE copolymer (melting point: 248° C., MFR: 30 g/10 min)
FEP: TFE/HFP copolymer (melting point: 265° C., MFR: 7 g/10 min)
Perfluororubber (FFKM): TFE/PMVE copolymer
Silicone rubber: KE-136Y-U available from Shin-Etsu Chemical Co., Ltd.
Chloroprene rubber (CR): Denka Chloroprene M-40 available from Denka Company Limited
Chlorosulfonated polyethylene rubber (CSM): Hypalon 40 available from DuPont

### Examples 1 to 6 and Comparative Examples 1 and 2

Sheets each having a thickness of 0.2 mm and a diameter of 120 mm were produced from the respective materials by compression molding using a heat press. PTFE was molded at a temperature that is 50° C. to 70° C. higher than the melting point and at a pressure of 5 MPa. Other resins were each molded at a temperature that is 40° C. higher than the melting point and at a pressure of 3 MPa. The rubbers were crosslink-molded by press vulcanization at a temperature of 100° C. to 200° C.

Using the resulting sheets, the long-term chemical resistance and the chemical permeability were evaluated by the following methods. Table 1 shows the results.

### <Long-Term Chemical Resistance (Organic Acid Resistance)>

A test piece (the above sheet) was immersed in an 80% aqueous solution of acetic acid at 50° C. for one month, and the appearance thereof was observed and evaluated based on the following criteria.

Good: No change in appearance (no change in appearance including discoloration, swelling, or cracks)

Acceptable: Slight change in appearance (slight change in appearance including discoloration, swelling, or cracks)

Poor: Significant change in appearance (significant change in appearance including discoloration, swelling, or cracks)

### <Chemical Permeability>

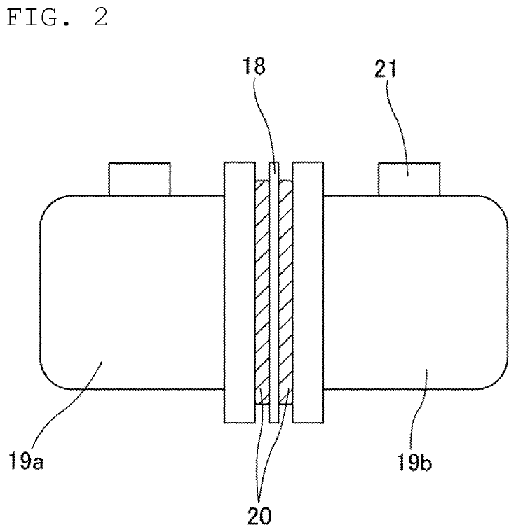

A sample sheet 18 (the above sheet) was sandwiched between two glass containers 19a and 19b (each having a capacity of 200 ml) shown in FIG. 2 using O-rings 20. The container 19a on one side of the sheet was charged with 200 ml of hydrochloric acid having a concentration of 35 mass % or nitric acid having a concentration of 60 mass %, and the container 19b on the other side was charged with 200 ml of pure water. The system was placed in a constant-temperature bath (the sample sheet 18 had a wetted surface of 70 mmφ). The system was left in such a state for 40 days, and a sample in an amount of about 1 ml was collected from a sampling port 21 of the container 19Sb containing pure water. The hydrochloric acid ion concentration or nitric acid ion concentration (Y ppm) of the sample was quantified by ion chromatography (IC7000-E available from Yokogawa Electric Corporation). The permeation amount of hydrochloric acid or nitric acid (X g·cm/cm²) was calculated using the following formula, and the permeation coefficient of hydrochloric acid or nitric acid was determined.

X=Y×200×0.02×10⁻⁶/(3.5×3.5×3.14)

TABLE 1

| | | | | | | | | Comparative | Comparative |
|---|---|---|---|---|---|---|---|---|---|
| | | Example 1 | Example 2 | Example 3 | Example 4 | Example 5 | Example 6 | Example 1 | Example 2 |
| Material | | PTFE | PFA | CPT | FFKM | FEP | Silicone | CR | CSM |
| | | | | | | | rubber | | |
| Evaluation item | | | | | | | | | |
| (1) Long-term chemical resistance | | Good | Good | Good | Good | Good | Acceptable | Poor | Poor |
| (2) Chemical | 35% HCl | 7.5 × 10⁻¹¹ | 1.5 × 10⁻¹¹ | 0.3 × 10⁻¹¹ | 4.0 × 10⁻¹¹ | 1.4 × 10⁻¹¹ | >1.0 × 10⁻⁹ | >1.0 × 10⁻⁹ | >1.0 × 10⁻⁹ |
| permeability | 60% HNO₃ | 5.7 × 10⁻¹² | 1.5 × 10⁻¹² | 0.4 × 10⁻¹² | 5.0 × 10⁻¹² | 1.3 × 10⁻¹² | >1.0 × 10⁻¹⁰ | >1.0 × 10⁻¹⁰ | >1.0 × 10⁻¹⁰ |
| (g · cm/cm² · sec) | | | | | | | | | |

| | | | | | | | | |
|---|---|---|---|---|---|---|---|---|
| Comprehensive evaluation | Good | Good | Good | Good | Good | Acceptable | Poor | Poor |

### REFERENCE SIGNS LIST

1: packing
10: livestock sensor
11: housing
12: battery
13: signal processing circuit
14: acceleration sensor
15: temperature sensor
16: fixed pH sensor
17: radio transmitter
18: sample sheet
19a, 19b: glass container
20: O-ring
21: sampling port

What is claimed is:

1. A seal for a livestock sensor, the seal comprising:
a resin, or
a rubber free from a chlorine atom,
wherein the seal is at least one selected from the group consisting of a packing, an O-ring, and a gasket, and
the seal is configured to prevent a body fluid containing an organic acid in livestock from entering a space that contains a detecting portion or a substrate of the livestock sensor.

2. The seal for a livestock sensor according to claim 1,
wherein the resin is a fluororesin.

3. The seal for a livestock sensor according to claim 2,
wherein the fluororesin includes at least one selected from the group consisting of polytetrafluoroethylene, a tetrafluoroethylene/perfluoro(alkyl vinyl ether) copolymer, a tetrafluoroethylene/hexafluoropropylene copolymer, a tetrafluoroethylene/perfluoroalkyl allyl ether copolymer, and a chlorotrifluoroethylene/tetrafluoroethylene/perfluoro(alkyl vinyl ether) copolymer.

4. The seal for a livestock sensor according to claim 1,
wherein the rubber is a fluorine-containing rubber.

5. The seal for a livestock sensor according to claim 4,
wherein the fluorine-containing rubber is a perfluororubber.

6. A livestock sensor comprising the seal for a livestock sensor according to claim 1.
